(19) United States
(12) Patent Application Publication
(10) Pub. No.: US 2024/0336928 A1
(43) Pub. Date: Oct. 10, 2024

(54) METHODS FOR IMPROVING SODIUM SALT TOLERANCE

(71) Applicant: Alliance for Sustainable Energy, LLC, Golden, CO (US)

(72) Inventors: Rebecca Ann WILKES, Lakewood, CO (US); Allison Jean ZIMONT WERNER, Denver, CO (US); Gregg Tyler BECKHAM, Golden, CO (US)

(21) Appl. No.: 18/627,252

(22) Filed: Apr. 4, 2024

Related U.S. Application Data
(60) Provisional application No. 63/494,184, filed on Apr. 4, 2023.

(51) Int. Cl. C12N 15/78; C07K 14/21; C12N 1/20; C12N 9/52; C12N 15/52; C12R 1/40

(52) U.S. Cl. CPC C12N 15/78; C07K 14/21; C12N 1/205; C12N 9/52; C12N 15/52; C12N 2510/00; C12R 2001/40

(57) ABSTRACT

Disclosed herein are five genes that were identified and engineered into Pseudomonas putida KT2440 and P. putida CJ781 to improve tolerance to sodium salt (Na+).

### CROSS-REFERENCE TO RELATED APPLICATIONS

This application claims priority under 35 U.S.C. § 119 to U.S. provisional patent application No. 63/494,184 filed on 4 Apr. 2023, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Engineering profitable titers is a current limitation to bioproduction of commodity chemicals, such as muconic acid and β-ketoadipic acid, from lignin-derived aromatic compounds. During fed-batch fermentation to produce diacids from aromatic acids, cells are exposed to increasing concentrations of Na+ added in the form of NaOH to maintain the pH of the media by neutralizing acids introduced both from the aromatic feedstock and the accumulation of product. The accumulation of Na+ in the media can subsequently interfere with cell growth by disrupting the ionic and osmotic balance in the cells. Improvements in sodium salt tolerance in native and production strains is an important step towards realization of cost-effective production of acidic chemicals. Overall, Na+ tolerance is important for any feedstock or product that contains acidic components, including aliphatic acids (such as acetic acid, lactic acid, succinic acid, citric acid, and levulinic acid), fatty acids, diacids (such as muconic acid and β-ketoadipic acid), amino acids (such as glutamic acid and aspartic acid), and aromatic acids (such as coumaric acid and ferulic acid).

Others have engineered Pediococcus acidilactici to overexpress native Na+ antiporters to increase production of sodium lactate from sugars. Similarly, Escherichia coli was previously engineered by others to increase sodium tolerance and lactate production from sugars by overexpressing nhaA and nhaR. Zymomonas mobilis has been previously engineered to increase sodium tolerance and ethanol production by overexpression of nhaA. None of this prior work uses native Na+ transporters from P. putida KT2440 nor does it address homologues of PP_4799 and PP_2714. Further, this work by others only reported improvement to the bioproduction of sodium lactate or ethanol by their respective species.

### SUMMARY

In an aspect, disclosed herein is a method for increasing the salt tolerance in a non-naturally occurring P. putida sp. comprising overexpressing genes selected from the group consisting essentially of PP_4799, PP_2714, PP_1132, PP_2444, and PP_4974.

In an aspect, disclosed herein is a method for increasing the production of a compound of interested comprising the step of increasing the salt tolerance in a non-naturally occurring P. putida sp. comprising overexpressing genes selected from the group consisting essentially of PP_4799, PP_2714, PP_1132, PP_2444, and PP_4974. In an embodiment, the compound of interest is muconic acid. In an embodiment, the compound of interest is 3-ketoadipic acid.

In an aspect, disclosed herein is a method for increasing the salt tolerance in a non-naturally occurring P. putida sp. when compared to a naturally occurring P. putida sp., the method comprising overexpressing at least one gene in the non-naturally occurring P. putida sp. selected from the group consisting essentially of PP_4799, PP_2714, PP_1132, PP_2444, and PP_4974.

In an embodiment, the method further comprises growth of the non-naturally occurring P. putida sp. in a growth media comprising salt concentrations from about 100 to 900 mM. In an embodiment, the non-naturally occurring P. putida sp. exhibits a 25 to 50 percent decrease lag time in growth in the media comprising salt concentrations from about 100 to 900 mM when compared to a naturally occurring P. putida sp. In an embodiment, the non-naturally occurring P. putida sp. exhibits a 25 to 50 percent decrease lag time in growth in the media comprising salt concentrations at about 900 mM when compared to a naturally occurring P. putida sp. In an embodiment, the non-naturally occurring P. putida sp. exhibits a 50 percent decrease lag time in growth in the media comprising salt concentrations at about 900 mM when compared to a naturally occurring P. putida sp. In an embodiment, the non-naturally occurring P. putida sp. exhibits a 50 percent decrease lag time in growth in the media comprising salt concentrations at about 900 mM when compared to a naturally occurring P. putida sp. and wherein PP_4799 is overexpressed in the non-naturally occurring P. putida sp. In an embodiment, the non-naturally occurring P. putida sp. exhibits about a 41 percent decrease lag time in growth in the media comprising salt concentrations at about 900 mM when compared to a naturally occurring P. putida sp. and wherein PP_1132 is overexpressed in the non-naturally occurring P. putida sp. In an embodiment, the non-naturally occurring P. putida sp. exhibits about a 25 percent decrease lag time in growth in the media comprising salt concentrations at about 900 mM when compared to a naturally occurring P. putida sp. and wherein PP_2444 is overexpressed in the non-naturally occurring P. putida sp.

In an aspect, disclosed herein is a method for increasing the production of a compound of interest comprising the step of increasing the salt tolerance in a non-naturally occurring P. putida sp., the method comprising overexpressing genes selected from the group consisting essentially of PP_4799, PP_2714, PP_1132, PP_2444, and PP_4974. In an embodiment, the compound of interest is muconic acid. In an embodiment, the non-naturally occurring P. putida sp. comprises a genotype of ΔcatRBCA::Ptac:catA ΔpcaHG::Ptac:aroY:ecdBD Δcrc ΔpobAR ΔfpvA::Ptac:praI:vanAB.

In another aspect, disclosed herein is a non-naturally occurring P. putida sp. capable of growing and producing muconic acid in media comprising salt concentrations from about 100 to 900 mM wherein the non-naturally occurring P. putida sp. comprises overexpression of a at least one gene selected from the group consisting essentially of PP_4799, PP_2714, PP_1132, PP_2444, and PP_4974. In an embodiment, the overexpression of the at least one gene results in the overexpression of at least one protein selected from the group consisting essentially of a muramoyltetrapeptide carboxypeptidase, a sensor protein QseC, a Na(+)/H(+) antiporter NhaA1, a transcriptional regulator in the LysR family, and a NhaP-type Na+(K+)/H+ antiporter. In an embodiment, the production rate of muconic acid is up to five times greater than the production rate of muconic acid under the same growth conditions of a non-naturally occurring P. putida strain that does not overexpress at least one gene selected from the group consisting essentially of PP_4799, PP_2714, PP_1132, PP_2444, and PP_4974. In an embodiment, the non-naturally occurring P. putida strain that does not overexpress at least one gene selected from the group consisting essentially of PP_4799, PP_2714, PP_1132, PP_2444, and PP_4974 comprises a genotype of ΔcatRBCA::Ptac:catA ΔpcaHG::Ptac:aroY:ecdBD Δcrc ΔpobAR ΔfpvA::Ptac:praI:vanAB. In an embodiment, the non-naturally occurring P. putida strain is capable of producing muconic acid from coumaric acid. In an embodiment, the non-naturally occurring P. putida strain is capable of producing muconic acid from coumaric acid in stoichiometric yields greater than 95%. In an embodiment, the non-naturally occurring P. putida strain is P. putida KT2440. In an embodiment, the P. putida strain is selected from the group consisting essentially of RW40, RW42, RW49, RW51, RW54, RW69, RW70, RW83, RW84, RW85 and RW86. In an embodiment, the non-naturally occurring P. putida sp. 12 further comprises a genotype of ΔcatRBCA::Ptac:catA ΔpcaHG::Ptac:aroY:ecdBD Δcrc ΔpobAR ΔfpvA::Ptac:praI:vanAB.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts improvements in lag and growth rate at increasing concentrations of sodium salt in P. putida KT2440 grown in BioscreenC. Growth rates and lag were determined from a Single Gompertz model. SN182 (P. putida KT2440 pBTL-2) was used as an empty vector control and SN34 (P. putida KT2440 pBTL-2_GFP) was a control for high expression of a protein not associated with Na+ tolerance. All data (mean±s.d.) were from replicates (n=3). Significance was determined by one-way ANOVA followed by Tukey HSD post hoc tests. Reported P-values are relative to SN182 control.

FIG. 2 depicts lag and growth rate comparison at increasing concentrations of sodium salt in P. putida CJ781 grown in BioscreenC. Growth rates and lag were determined from a Single Gompertz model. The parent strain CJ781 was used as the control for the six engineered strains with chromosomal integration of Plac or Ptac to increase expression of target genes. All data (mean±s.d.) were from replicates (n=3). Significance was determined by one-way ANOVA followed by Tukey HSD post hoc tests. Reported P-values are relative to CJ781 control. Significant improvements in lag relative to CJ781 were found for RW83, RW84, RW85, and RW89 grown on glucose and 604 mM Na+.

FIGS. 3A and 3B depict a comparison of CJ781 and engineered strains in shake flask during production of muconate from coumarate. A) Dynamics of glucose and coumarate consumption, intermediate accumulation, and muconate production in CJ781 and engineered strains. B) Average and standard deviation of bacterial growth (OD600) and muconate production (mM) after 25 h for CJ781 compared to the engineered strains. All data (mean±s.d.) were from replicates (n=3). Both RW89 and RW84 had faster growth and muconate production relative to the parent strain CJ781.

### DETAILED DESCRIPTION

Disclosed herein are methods and compositions of matter for five genes that were identified and engineered into Pseudomonas putida KT2440 and P. putida CJ781 to improve the native tolerance to sodium salt (Na+). Both PP_4799 and PP_2714 were selected to be overexpressed based on a RB-TnSeq study. The remaining three genes (PP_1132, PP_2444, and PP_4974) were overexpressed due to their involvement in Na+ transmembrane transportation.

In a prophetic embodiment, genes associated with sodium salt tolerance in P. putida KT2440 can be identified by using RB-TnSeq to identify genetic targets for improved tolerance of P. putida towards compounds relevant to lignin conversion. In an embodiment, enhanced sodium tolerance to support bioproduction can be achieved through using the methods and compositions of matter disclosed herein. In an embodiment, P. putida strain CJ781 served as the parent strain for all the chromosomal integrations of the target genes. CJ781 contains pathways for production of muconic acid at stoichiometric yields (100%) from coumaric acid.

In an embodiment, Pseudomonas putida KT2440 was engineered to produce five new strains that were found to improve the growth phenotype under Na+ stress (Table 1). For all the strains with wild-type as the parent strain, genes were expressed on the pBTL-2 plasmid using the lac promoter. An optimized ribosomal binding site and the target gene were integrated between the XbaI and EcoRV sites of pBTL-2. Subsequently, P. putida CJ781 was engineered for overexpression of these five target genes using chromosomal integration to produce an additional six strains (Table 1). For all the strains with P. putida CJ781 as the parent strain, chromosomal integration of tonB termination site, a lac or tac promoter, and an optimized ribosomal binding site was conducted using pK18sB backbone with homology regions. The genotype of CJ781 is P. putida KT2440 ΔcatRBCA::P_{tac}:catA ΔpcaHG::P_{tac}:aroY:ecdBD Δcrc ΔpobAR ΔfpvA::P_{tac}:praI:vanAB.

All pBTL-2 plasmids were ordered from TWIST while the pK18sB plasmids were cloned using Gibson assembly. All chromosomal integrations were sequenced confirmed.

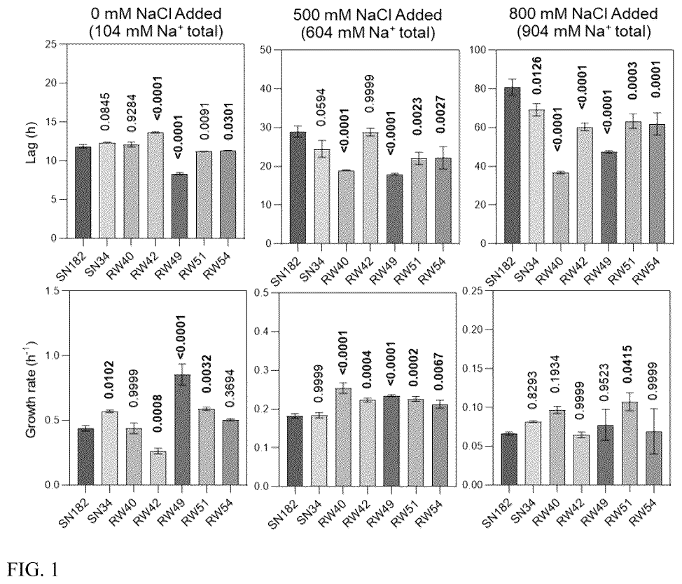

TABLE 1
Strains identified to have increased tolerance to sodium salt.

| | | | Phenotype relative to empty vector |
|---|---|---|---|
| Strain | Genotype | Overexpressed Protein | control (also see FIG. 1) |
| RW40 | P. putida KT2440 | Putative | Significant improvement in lag and |
| | pBTL-2_Plac:PP_4799 | muramoyltetrapeptide | moderate increase in growth rate at |
| | | carboxypeptidase; | both 604 mM and 904 mM Na⁺ |
| | | plasmid expression | concentrations. |
| RW42 | P. putida KT2440 | Sensor protein QseC; | Significant improvement in lag at 604 |
| | pBTL-2_Plac:PP_2714 | plasmid expression | mM and 904 mM Na⁺ concentrations |
| RW49 | P. putida KT2440 | Na(+)/H(+) antiporter | Significant improvement in lag and |
| | pBTL-2_Plac:nhaA-I | NhaA1 (PP_1132); | moderate increase in growth rate at |
| | | plasmid expression | 604 mM and 904 mM Na⁺ |
| | | | concentrations |
| RW51 | P. putida KT2440 | Transcriptional regulator, | Significant improvement in lag and |
| | pBTL-2_Plac:PP_2444 | LysR family; plasmid | growth rate at 604 mM and 904 mM |
| | | expression | Na⁺ concentrations |
| RW54 | P. putida KT2440 | NhaP-type Na+(K+)/H+ | Significant improvement in lag at 604 |
| | pBTL-2_Plac:PP_4974 | antiporter; plasmid | mM and 904 mM Na⁺ concentrations |
| | | expression | |
| RW69 | P. putida CJ781 | Putative | No improvement compared to CJ781 |
| | Plac:PP_4799 | muramoyltetrapeptide | base strain. Higher expression likely |
| | | carboxypeptidase; | needed. |
| | | genome integration Plac | |
| | | promoter | |
| RW70 | P. putida CJ781 | Sensor protein QseC; | No improvement compared to CJ781 |
| | Plac:PP_2714 | genome integration Plac | base strain. Higher expression likely |
| | | promoter | needed. |
| RW83 | P. putida CJ781 | Transcriptional regulator, | Significant improvement in lag at 604 |
| | Plac:nhaR | LysR family; genome | mM Na⁺ and some improvement at 904 |
| | | integration Plac promoter | mM Na⁺ compared to CJ781 base |
| | | | strain. |
| RW84 | P. putida CJ781 | NhaP-type Na+(K+)/H+ | Significant improvement in lag at 604 |
| | Plac:nhaP | antiporter; genome | mM Na⁺ and some improvement at 904 |
| | | integration Plac promoter | mM Na⁺ compared to CJ781 base |
| | | | strain. Faster muconate production in |
| | | | shake flask relative to CJ781. |
| RW85 | P. putida CJ781 | Na(+)/H(+) antiporter | Significant improvement in lag at 604 |
| | Plac:nhaI | NhaA1 (PP_1132); | mM Na⁺ compared to CJ781 base |
| | | genome integration Plac | strain. |
| | | promoter | |
| RW89 | P. putida CJ781 | Putative | Significant improvement in lag at 604 |
| | Ptac:PP_4799 | muramoyltetrapeptide | mM Na⁺ compared to CJ781 base |
| | | carboxypeptidase; | strain. Faster muconate production in |
| | | genome integration Ptac | shake flask compared to CJ781. |
| | | promoter (higher | |
| | | expression) | |

TABLE 2
BLASTP analysis of NhaA-I and NhaP from P. putida used as disclosed
herein when compared to NhaA from E. coli and Z. mobilis reported
to improve sodium tolerance in previous studies.

| | P. putida | P. putida |
|---|---|---|
| | KT2440 NhaA-I | KT2440 NhaP |
| Percent Identity (%) | (PP_1132) | (PP_4974) |
| E. coli NhaA | 56.95% | No significant similarity |
| Z. mobilis NhaA | 39.64% | No significant similarity |
| (ZMO0119) | | |

Enhanced sodium salt tolerance in P. putida KT2440 was determined by calculating the growth rate and lag of the five strains compared to the pBTL-2 empty vector control (SN182) during growth at three Na+ concentrations (104 mM, 604 mM, and 904 mM) in the BioscreenC (FIG. 1). All five strains were grown in M9 minimal medium (pH 7.0, 6.78 g/L Na2HPO₄, 3 g/L KH₂PO₄, 0.5 g/L NaCl, 1 g/L NH₄Cl, 2 mM MgSO₄, 100 μM CaCl₂), and 18 μM FeSO₄) supplemented with 20 mM glucose as the carbon source and 50 μg/mL kanamycin to maintain the plasmid. To examine the growth phenotype under osmotic stress from Na+, either 500 mM NaCl or 800 mM NaCl was added to the growth media.

In an embodiment, lag time (also referred to herein as “lag”) is defined as the initial period in the life of a bacterial population when cells are adjusting to a new environment before starting exponential growth. The greatest improvement in Na+ tolerance was found for RW40, which had a 50% decrease in the lag time compared to the control at the highest Na+ concentration. With up to a 41% decrease in lag compared to the control, RW49 was the next best strain for improved tolerance to Na+. Moderate improvements, around a 25% decrease in lag relative to the control, were observed for RW42, RW51, and RW54 (FIG. 1). All the overexpression strains were found to significantly improve growth rate at 604 mM Na+, but only RW51 significantly increased the growth rate at the highest Na+ concentration (FIG. 1). Overall, while the moderate improvements in growth rate suggest that these overexpression strains may support glucose catabolism, the consistent improvement in lag demonstrates that all five strains exhibit increased tolerance to Na+ stress.

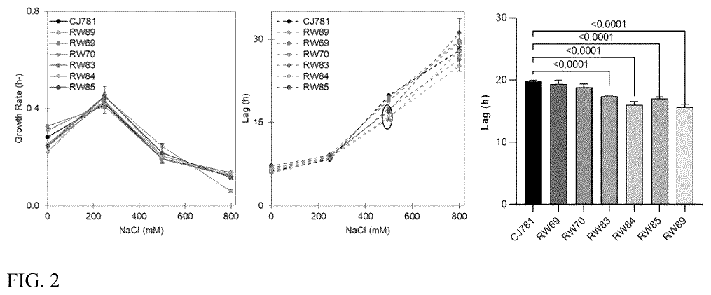

In addition to testing plasmid-based expression, the chromosomal integration of lac or tac promoters for stable high expression of the above target genes was evaluated in the muconate production strain P. putida CJ781. The engineered strains were first grown in BioscreenC in the media as described above with the exclusion of kanamycin. Four different Na+ concentrations were tested (104 mM, 354 mM, 604 mM, and 904 mM) to assess Na+ tolerance. At 604 mM Na+, RW83, RW84, RW85, and RW89 exhibited significantly improved lags compared to CJ781 (FIG. 2). However, there was no significant improvement in lag at the lower or higher concentrations of Na+ or in growth rates compared to CJ781, indicating that a different promoter to mimic the expression level from the plasmid-based expression could improve these results further (FIG. 2).

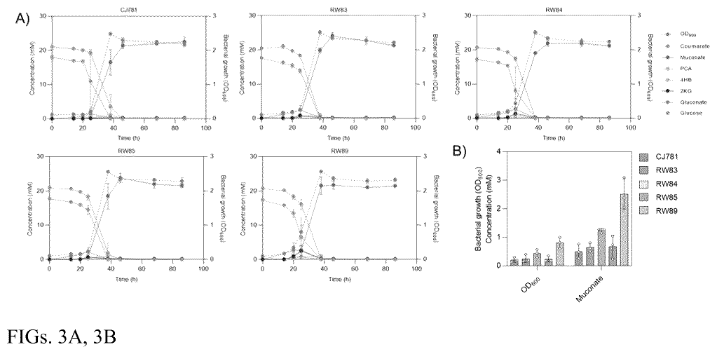

As cellular growth of CJ781 in fed-batch fermentation plateaus at 465 mM Na+, the four strains that showed improvement in the BioscreenC (RW83, RW84, RW85, and RW89) were analyzed during growth in shake flasks with 20 mM glucose, 20 mM coumarate, and 250 mM NaCl in a 2× M9 media (total of 462 mM Na+). During growth in shake flask, RW89 exhibited the fastest production of muconate (5-fold greater than CJ781 at 25 h) and the fastest growth (4-fold greater than CJ781 at 25 h) (FIG. 3). The second-best strain was RW84, which had a 2.4-fold greater muconate production and 2-fold faster growth relative to CJ781 at 25 h (FIG. 3). While not a significant improvement in muconate production, there was a 1.3-fold higher concentration of muconate for RW83 and RW85 at 25 h compared to CJ781. These small improvements in shake flask could represent substantial improvements during growth in bioreactors. Currently, to further improve Na+ tolerance, the four overexpression targets that showed improvements in lag relative to the parent strain CJ781 will be stacked through chromosomal integration in combinations of two, three, and all four genes.

In an embodiment, the methods disclosed above is useful for the production of muconic acid.

The foregoing disclosure has been set forth merely to illustrate the invention and is not intended to be limiting.

We claim:

1. A method for increasing the salt tolerance in a non-naturally occurring P. putida sp. when compared to a naturally occurring P. putida sp., the method comprising overexpressing at least one gene in the non-naturally occurring P. putida sp. selected from the group consisting essentially of PP_4799, PP_2714, PP_1132, PP_2444, and PP_4974.

2. The method of claim 1 further comprising growth of the non-naturally occurring P. putida sp. in a growth media comprising salt concentrations from about 100 to 900 mM.

3. The method of claim 2 wherein the non-naturally occurring P. putida sp. exhibits a 25 to 50 percent decrease lag time in growth in the media comprising salt concentrations from about 100 to 900 mM when compared to a naturally occurring P. putida sp.

4. The method of claim 1 wherein the non-naturally occurring P. putida sp. exhibits a 25 to 50 percent decrease lag time in growth in the media comprising salt concentrations at about 900 mM when compared to a naturally occurring P. putida sp.

5. The method of claim 1 wherein the non-naturally occurring P. putida sp. exhibits a 50 percent decrease lag time in growth in the media comprising salt concentrations at about 900 mM when compared to a naturally occurring P. putida sp.

6. The method of claim 1 wherein the non-naturally occurring P. putida sp. exhibits a 50 percent decrease lag time in growth in the media comprising salt concentrations at about 900 mM when compared to a naturally occurring P. putida sp. and wherein PP_4799 is overexpressed in the non-naturally occurring P. putida sp.

7. The method of claim 1 wherein the non-naturally occurring P. putida sp. exhibits about a 41 percent decrease lag time in growth in the media comprising salt concentrations at about 900 mM when compared to a naturally occurring P. putida sp. and wherein PP_1132 is overexpressed in the non-naturally occurring P. putida sp.

8. The method of claim 1 wherein the non-naturally occurring P. putida sp. exhibits about a 25 percent decrease lag time in growth in the media comprising salt concentrations at about 900 mM when compared to a naturally occurring P. putida sp. and wherein PP_2444 is overexpressed in the non-naturally occurring P. putida sp.

9. A method for increasing the production of a compound of interest comprising the step of increasing the salt tolerance in a non-naturally occurring P. putida sp., the method comprising overexpressing genes selected from the group consisting essentially of PP_4799, PP_2714, PP_1132, PP_2444, and PP_4974.

10. The method of claim 9 wherein the compound of interest is muconic acid.

11. The method of claim 9 wherein the non-naturally occurring P. putida sp. comprises a genotype of ΔcatRBCA::P_{tac}:catA ΔpcaHG::P_{tac}:aroY:ecdBD Δcrc ΔpobAR ΔfpvA::P_{tac}:praI:vanAB.

12. A non-naturally occurring P. putida sp. capable of growing and producing muconic acid in media comprising salt concentrations from about 100 to 900 mM wherein the non-naturally occurring P. putida sp. comprises overexpression of a at least one gene selected from the group consisting essentially of PP_4799, PP_2714, PP_1132, PP_2444, and PP_4974.

13. The non-naturally occurring P. putida sp. of claim 12 wherein the overexpression of the at least one gene results in the overexpression of at least one protein selected from the group consisting essentially of a muramoyltetrapeptide carboxypeptidase, a sensor protein QseC, a Na(+)/H(+) antiporter NhaA1, a transcriptional regulator in the LysR family, and a NhaP-type Na+(K+)/H+ antiporter.

14. The non-naturally occurring P. putida sp. of claim 12 wherein the production rate of muconic acid is up to five times greater than the production rate of muconic acid under the same growth conditions of a non-naturally occurring P. putida strain that does not overexpress at least one gene selected from the group consisting essentially of PP_4799, PP_2714, PP_1132, PP_2444, and PP_4974.

15. The non-naturally occurring P. putida sp. of claim 14 wherein the non-naturally occurring P. putida strain that does not overexpress at least one gene selected from the group consisting essentially of PP_4799, PP_2714, PP_1132, PP_2444, and PP_4974 comprises a genotype of ΔcatRBCA::P_{tac}:catA ΔpcaHG::P_{tac}:aroY:ecdBD Δcrc ΔpobAR ΔfpvA::P_{tac}:praI:vanAB.

16. The non-naturally occurring P. putida strain of claim 12 capable of producing muconic acid from coumaric acid.

17. The non-naturally occurring P. putida strain of claim 12 capable of producing muconic acid from coumaric acid in stoichiometric yields greater than 95%.

18. The non-naturally occurring P. putida strain of claim 12 wherein the P. putida strain is P. putida KT2440.

19. The non-naturally occurring P. putida strain of claim 12 wherein the P. putida strain is selected from the group consisting essentially of RW40, RW42, RW49, RW51, RW54, RW69, RW70, RW83, RW84, RW85 and RW86.

20. The non-naturally occurring P. putida sp. of claim 12 further comprising a genotype of ΔcatRBCA::P_{tac}:catA ΔpcaHG::P_{tac}:aroY:ecdBD Δcrc ΔpobAR ΔfpvA::P_{tac}:praI:vanAB.
